(19) United States
(12) Patent Application Publication
(10) Pub. No.: US 2005/0234405 A1
(43) Pub. Date: Oct. 20, 2005

(54) Site securement device for securing intravascular tubing

(76) Inventors: W. Cary Dikeman, Leawood, KS (US); Steve Cash, St. Charles, IL (US)

Correspondence Address: BLACKWELL SANDERS PEPER MARTIN LLP, 4801 Main Street, Suite 1000, KANSAS CITY, MO, 64112, US

(21) Appl. No.: 10/826,747

(22) Filed: Apr. 16, 2004

(51) Int. Cl. A61M 5/32

(52) U.S. Cl. 604/180; 128/DIG. 6; 128/DIG. 26

(57) ABSTRACT

A site securement device for use in medical infusion therapy to selectively and temporarily retain a section of intravascular (IV) tube. The present device includes a unitary body with uniform or non-uniform channels for temporarily receiving and retaining a section of IV tubing in one or more channels, and may include an adhesive pad attached thereto for easy attachment to the skin of a patient.

### FIELD OF THE INVENTION

This invention relates to medical devices and more specifically to site securement devices used for medical infusion therapy.

### BACKGROUND OF THE INVENTION

The medical community relies heavily on medical infusion therapy to deliver pharmaceuticals, blood products and other fluids to patients. Medical infusion therapy uses peripheral and central intravascular devices, including venous and arterial catheters, to tap into veins and arteries, often for extended periods of time. These intravascular devices must be secured to the patient at the insertion site to prevent moving or dislodging the catheter.

The common way to secure intravascular (IV) tubing is by taping the tubing to the patient. The tape usually comes from a nurse's pocket, where contamination is a problem. The tape introduces germs to the insertion site, where sterility is crucial. If the intravascular device becomes infected, the whole apparatus must be replaced to avoid harming the patient. This requires extra time on the part of the nurse to replace the apparatus, costs the hospital and patient because new catheters and tubing are required, and causes the patient physical discomfort as the apparatus is replaced.

Another problem with current ways to secure IV tubing is the lack of flexibility and extra time required to change the position of the tubing. IV tubing may be re-positioned to increase the patient's comfort, make room for other medical infusion devices, or adjust the tubing while gravity supply sources are replaced. Any time the tubing must be re-positioned, the nurse must take extra time to remove the previous site securement means, such as tape, and then re-secure the tubing. This sequence of events requires precious nursing attention that could be better spent on other aspects of patient care.

### BRIEF SUMMARY OF THE INVENTION

There is, therefore, provided in the practice of the present invention a novel site securement device for selectively restraining intravascular tubing and securing the tubing to the patient. The site securement device includes a unitary body with multiple channels. The site securement device can be used in medical infusion therapy to quickly and hygienically secure IV tubing to a patient.

In a preferred embodiment, a site securement device includes a unitary body with multiple, uniform channels shaped to removably receive and hold a section of intravascular tubing. The multiple channels allow for efficient, sterile securement of the tubing and flexible placement of the tubing.

In another embodiment, the site securement device includes a unitary body with multiple channels where the channels vary in size to fit tubing with varying diameters/circumferences. The channels of varying size allow for multiple tubing configurations.

Accordingly, it is an object of the present invention to provide an improved site securement device for use in medical infusion therapy to selectively secure IV tubing without introducing contaminated elements.

It is a further object of the present invention to provide an improved site securement device with greater flexibility to allow for greater tube placement configurations.

### BRIEF DESCRIPTION OF THE DRAWINGS

These and other inventive features, advantages, and objects will appear from the following Detailed Description when considered in connection with the accompanying drawings in which similar reference characters denote similar elements.

FIG. 1 is a front elevational view of a site securement device constructed according to the teachings of the present invention.

FIG. 2 is a top view of the site securement device shown in FIG. 1.

FIG. 3 is a front elevational view of a site securement device similar to the device shown in FIG. 1 with an intravascular tube positioned in one channel.

FIG. 4 is a top view of another embodiment of a site securement device with non-uniform channels.

FIG. 5 is a front elevational view of a 6-channel site securement device.

FIG. 6 is a front elevational view of a spaced-channel site securement device.

FIG. 7 is a top view of a site securement device positioned on an adhesive pad.

FIG. 8 is a top view of a medical infusion therapy apparatus secured to one embodiment of the present site securement device and an adhesive pad.

### DETAILED DESCRIPTION

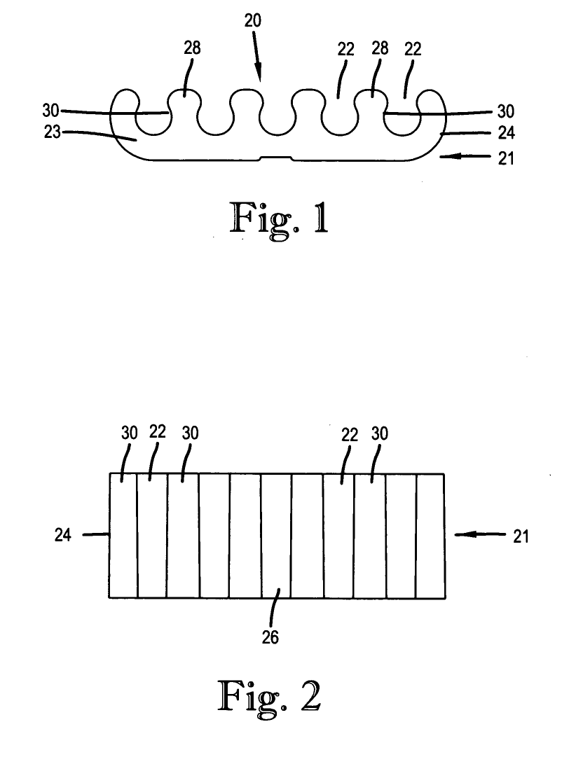

Referring to the drawings in greater detail, FIGS. 1 and 2 identify one embodiment of a site securement device 20 constructed in accordance with a preferred embodiment of the present invention. The site securement device 20 includes a unitary body 21 with multiple channels 22. The site securement device is operable to efficiently secure and re-position intravascular tubing for use in medical infusion therapy.

In a preferred embodiment, the site securement device 20, as best shown in FIG. 2, has a substantially rectangular-shaped, unitary body 21. In a preferred embodiment, the site securement device 20 is constructed from sterile plastic. The unitary body 21 has substantially straight sides 24 and 26, as shown in FIGS. 1 and 2.

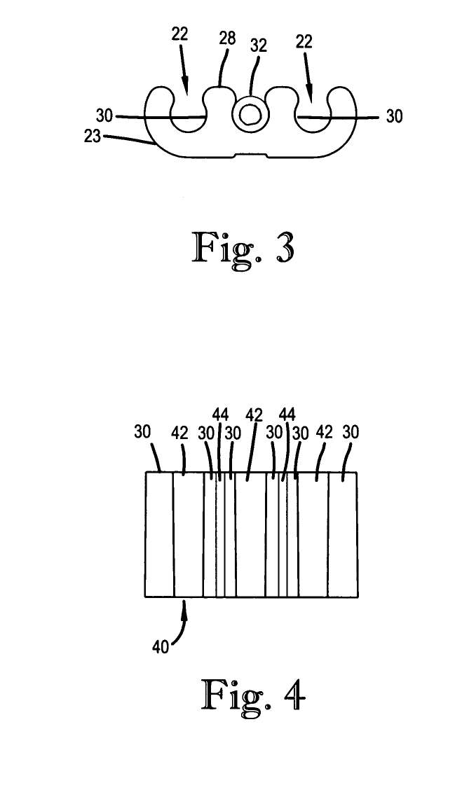

As illustrated in FIGS. 1 and 2, the site securement device 20 has multiple channels 22 for receiving intravascular (IV) tubing. The channels 22 are generally curved to follow the shape or contour of the IV tubing. The channels 22 extend the full length of the sides 24 but do not extend to the bottom of the device 20, leaving a base 23 extending the full length of the sides 26, as best shown in FIG. 1. The channels 22 extend around the sides of the tubing, such as tubing 32, covering more than half the side of the tubing 32 as shown in FIG. 3. The partition 30 between each channel 22 terminates in a flared, rounded end piece 28. The rounded end piece 28 helps guide the tubing into the channel as the nurse applies pressure to snap the tubing into the channel and also functions to hold the tubing within the channel. This design securely holds the tubing within a channel and prevents accidental removal of the tubing from the site securement device. When a nurse or other health care practitioner wishes to insert a section of IV tubing into the site securement device 20, the nurse selects the desired channel 22 and simply snaps the tubing into the channel. The tubing may be removed from the site securement device 20 by gently pulling the tubing from the channel. The same tubing may be returned and/or respositioned to the same channel or to a different channel depending on the needs of the patient. Alternatively, a different tube may be placed in a channel of the site securement device.

In an alternate embodiment, the site securement device includes a unitary body with multiple channels of non-uniform size. As shown in FIG. 4, the site securement device 40 includes channels 42 and 44, where channels 42 are larger than channels 44. In one embodiment, the outer channels are larger than the inner channels, although the arrangement of the channels may be altered to provide smaller outer channels or alternating channels. This design allows the site securement device to be compatible with a wide variety of multiple sizes of tubing. In another embodiment, the site securement device includes channels of multiple size where no two channels are the same size.

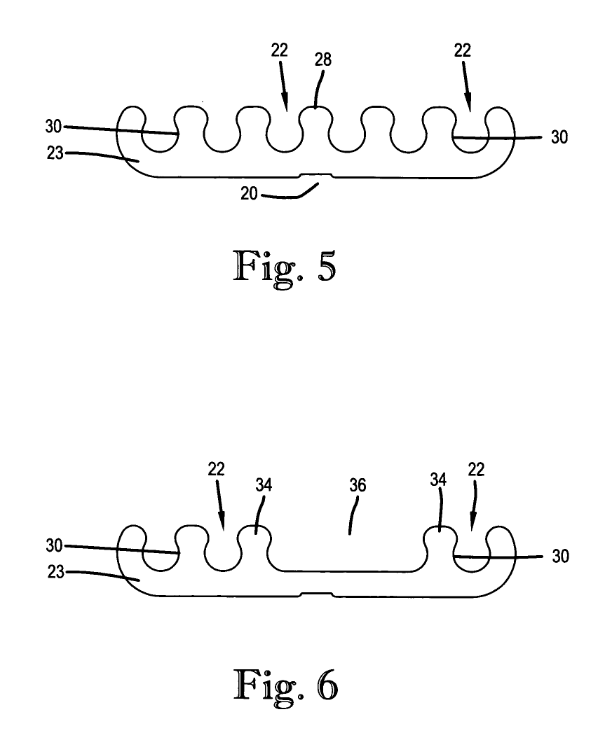

The number of channels in each site securement device may vary depending on the needs of the patient. As shown in FIG. 5, the device may include a six channel configuration. Alternatively, the present devices may include any plurality of channels depending upon the particular application.

Alternatively, as shown in FIG. 6, another embodiment 60 of the present site securement device may include multiple channels 22 in a unitary body with a space or spacer 36 positioned between two of the channels 34. The spacer 36 is not shaped or otherwise designed to receive IV tubing. In another embodiment, the terminal end portion of each partition 30 may be flared, but not rounded. It is also recognized and anticipated that some of the end portions of the various partitions 30 may be flared, but not rounded, while some may be flared and rounded.

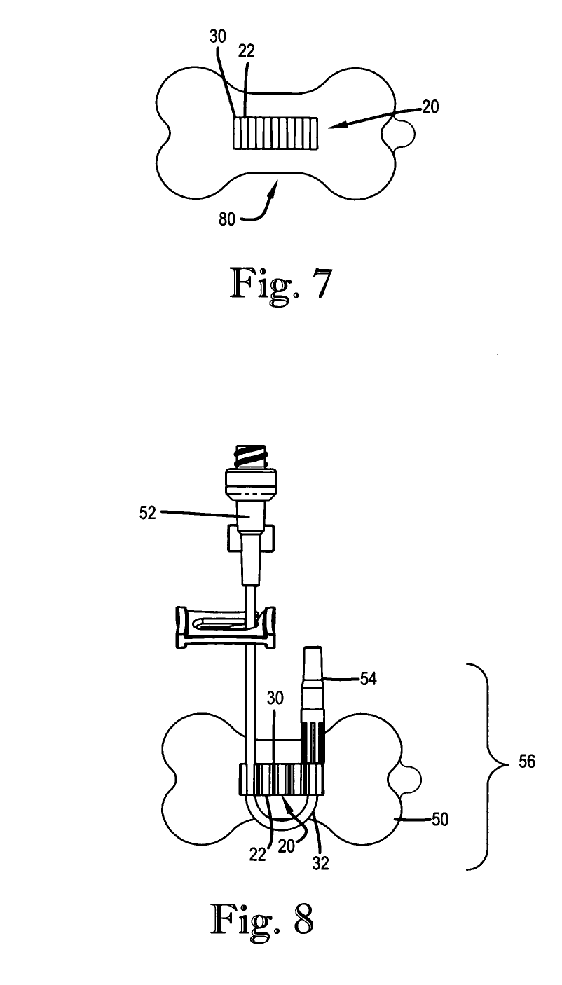

As shown in FIG. 7, the present site securement device 20 is attached to a patient using an adhesive pad 50. The adhesive pad 50 is generally rectangular in shape and may be sized to accommodate any number of site securement devices 20, including a single device 20, or the pad 50 may be sized larger to attach other parts of the infusion therapy apparatus. The adhesive pad 50 is a textured, sterile cloth pad with adhesive backing. The adhesive backing must be tacky enough to adhere to the skin of a patient for an extended period, but not so tacky that it requires a solvent or soap and water to remove the pad 50. It is also recognized and anticipated that the adhesive pad 50 may take on a wide variety of different shapes and sizes depending upon the particular application.

As shown in FIG. 8, the site securement device 20 may be used as part of a medical infusion therapy apparatus 56. The site securement device 20 is positioned on the adhesive pad 50. An intravenous tube 32 connects the catheter 52 to the fluid supply tube 54, which leads to a fluid reservoir (not shown). The tubing 32 is placed into selective channels 22 of the site securement device 20 as illustrated. Depending upon the particular infusion therapy apparatus 56 and the flexibility of its associated tubing, the tubing may be positioned in different channels 22 to achieve different configurations depending upon the particular geometry of the insertion site, the fluid source, the patient's position, and other parameters. Also, importantly, the present site securement device can be provided with the adhesive pad 50 in a sterile package. This enables a nurse to quickly remove the present device from a sterile package and immediately apply and secure the present device to a patient more efficiently and with less contamination than the current practice.

Thus, an improved site securement device is disclosed which utilizes a novel configuration of channels to selectively and temporarily secure an intravascular tube to a patient. This invention allows for superior patient care by providing a flexible, sterile and efficient site securement device for temporarily securing intravenous tubing to a patient. While preferred embodiments and particular applications of this invention have been shown and described, it is apparent to those skilled in the art that many other modifications and applications of this invention are possible without departing from the spirit and scope of the inventive concepts disclosed herein.

1. A site securement device comprising a unitary body including multiple channels of uniform shape and size, suitable for temporarily retaining a section of intravascular tubing in one or more of the channels.

2. The device according to claim 1 wherein the unitary body includes partitions between the channels with flared end pieces.

3. The device according to claim 2 wherein the end pieces are rounded.

4. The device according to claim 1 wherein the multiple channels include at least four channels.

5. The device according to claim 1 wherein at least two of the channels are separated by a space not adapted for temporarily retaining a section of intravascular tubing.

6. The device according to claim 1 including an adhesive pad attached to the site securement device for attachment to a patient.

7. A site securement device comprising a unitary body including multiple channels of uniform shape but varying size, suitable for temporarily retaining a section of intravascular tubing in one or more of the channels.

8. The device according to claim 7 wherein the unitary body includes partitions between the channels with flared end pieces.

9. The device according to claim 8 wherein the end pieces are rounded.

10. The device according to claim 7 wherein the multiple channels include at least four channels.

11. The device according to claim 7 wherein the device includes identical numbers of channels of each size.

12. The device according to claim 7 wherein the channels are configured to include outer channels of smaller size and inner channels of larger size.

13. The device according to claim 7 wherein the channels are configured to include outer channels of larger size and inner channels of smaller size.

14. The device according to claim 7 wherein at least two channels are separated by a space not adapted for temporarily retaining a section of intravascular tubing.

15. A site securement device comprising a unitary body including multiple channels of uniform shape but varying size, suitable for temporarily retaining a section of intravascular tubing in one or more of the channels, where no two channels are of the same size.

16. The device according to claim 15 wherein the unitary body includes partitions between the channels with flared end pieces.

17. The device according to claim 16 wherein the end pieces are rounded.

18. A medical infusion apparatus comprising a catheter connected to a fluid supply tube by a section of intravascular tubing, a site securement device for anchoring the catheter and fluid supply tube to a patient, said site securement device including a unitary body having multiple uniform channels for temporarily receiving a section of the intravascular tubing in one or more of the channels, and an adhesive pad attached to said site securement device, said adhesive pad being attachable to the skin of a patient.

19. A medical infusion apparatus comprising a catheter connected to a fluid supply tube by a section of intravascular tubing, a site securement device for anchoring the catheter and fluid supply tube to a patient, said site securement device including a unitary body having multiple uniformly shaped channels of varying sizes for temporarily receiving a section of the intravascular tubing in one or more of the channels, and an adhesive pad attached to said site securement device, said adhesive pad being attachable to the skin of a patient.

20. A site securement device comprising a unitary body having a plurality of channels of uniform shape and size, each channel being capable of temporarily retaining a section of intravascular tubing, and an adhesive pad attached to the site securement device for attachment to a patient.

21. A site securement device comprising a unitary body having a plurality of channels of uniform shape but varying size, each channel being capable of temporarily retaining a section of intravascular tubing, and an adhesive pad attached to the site securement device for attachment to a patient.
